(19) United States
(12) Patent Application Publication
(10) Pub. No.: US 2025/0255663 A1
(43) Pub. Date: Aug. 14, 2025

(54) TISSUE ABLATION DEVICE WITH NERVE STIMULATION FEATURE

(71) Applicant: Medtronic Xomed LLC, Minneapolis, MN (US)

(72) Inventor: James Britton Hissong, Jacksonville, FL (US)

(21) Appl. No.: 19/044,256

(22) Filed: Feb. 3, 2025

Related U.S. Application Data
(60) Provisional application No. 63/552,229, filed on Feb. 12, 2024.

(51) Int. Cl. A61B 18/12; A61B 5/00; A61B 5/294; A61B 18/00; A61B 18/14; A61N 1/05

(52) U.S. Cl. CPC A61B 18/1233; A61B 5/294; A61B 5/6852; A61B 18/1492; A61B 2018/00083; A61B 2018/00577; A61B 2018/00839; A61N 1/0551

(57) ABSTRACT

An ablation device includes an elongated shaft extending distally from a handle, a stimulation electrode, an ablation electrode, and an insulative ring. The stimulation electrode is disposed on a distal tip portion of the elongated shaft and is configured to electrically stimulate nerves. The ablation electrode is disposed on the elongated shaft and is axially spaced proximally from the stimulation electrode. The ablation electrode is configured to deliver electrosurgical energy to tissue. The insulative ring surrounds the elongated shaft and is disposed between the stimulation electrode and the ablation electrode to electrically isolate the stimulation electrode from the ablation electrode.

### CROSS-REFERENCE TO RELATED APPLICATION

This application claims the benefit of and priority to U.S. Provisional Patent Application No. 63/552,229 filed Feb. 12, 2024, the entire disclosure of which is incorporated by reference herein.

### FIELD

This disclosure relates to intraoperative neural monitoring during a tissue ablation procedure. More specifically, the disclosure relates to a tissue ablation system including an ablation device configured to ablate tissue and stimulate nerves.

### BACKGROUND

Ablation of benign thyroid nodules and some cancerous tumors typically involves inserting an energy-based ablation device (e.g., radiofrequency (RF), microwave, cryogenic, or laser device) into the nodules/tumors and causing cell death through heating. To avoid general anesthesia, hypothyroidism, and producing a scar from an incision at the front of the neck to remove the thyroid nodule/tumor, a percutaneous electrode may be inserted at the isthmus of the neck and directed/guided into the nodule/tumor by visualization with an ultrasound probe on the neck viewing the thyroid, the nodule/tumor, and the electrode progression through the tissue. Once the ablating portion of the electrode is within the target nodule/tumor, energy is applied, heating the tissue to levels that cause cell death/necrosis. One particular energy delivery approach is known as a “moving shot” technique, which is employed in modern thyroid ablation procedures for various reasons such as, for example, the thyroid's small size, the thyroid's close proximity to nerves and other important vessels, and the relatively faster speed at which ablation is performed using the “moving shot” technique since it is performed in awake patients. While there are benefits to this type of surgical “removal” of tissue, there remains risk to the nerves near the thyroid, in particular, the recurrent laryngeal nerve (RLN) and the vagus nerve, which are not generally visible in an ultrasound image. Additionally, the “moving shot” technique involves moving the electrode while ablating, which makes it difficult to ensure the safety of the procedure. It would be beneficial to know prior to initiating ablation that the ablation electrode is not in close proximity to the nerve such that the nerve could be damaged from heating.

### SUMMARY

Provided in accordance with aspects of the present disclosure is an ablation system including an ablation device configured to be inserted into an ablation target. The ablation device includes an elongated shaft extending distally from a handle. A stimulation electrode is disposed on a distal tip portion of the elongated shaft and is configured to electrically stimulate nerves. An ablation electrode is disposed on the elongated shaft and is axially spaced proximally from the stimulation electrode. The ablation electrode is configured to deliver electrosurgical energy to tissue. An insulative ring surrounds the elongated shaft and is disposed between the stimulation electrode and the ablation electrode. The ablation system also includes an electrosurgical generator configured to deliver electrosurgical energy to the ablation electrode of the ablation device and a nerve monitoring unit configured to deliver electrical stimulation signals to the stimulation electrode of the ablation device.

In an aspect of the present disclosure, the ablation system also includes a patient interface unit electrically coupleable to the nerve monitoring unit and configured to electrically couple the stimulation electrode to the nerve monitoring unit.

In another aspect of the present disclosure, the electrosurgical generator and the nerve monitoring unit are disposed within a common enclosure.

Also provided in accordance with aspects of the present disclosure is an ablation device including an elongated shaft extending distally from a handle, a stimulation electrode, an ablation electrode, and an insulative ring. The stimulation electrode is disposed on a distal tip portion of the elongated shaft and is configured to electrically stimulate nerves. The ablation electrode is disposed on the elongated shaft and is axially spaced proximally from the stimulation electrode. The ablation electrode is configured to deliver electrosurgical energy to tissue. The insulative ring surrounds the elongated shaft and is disposed between the stimulation electrode and the ablation electrode to electrically isolate the stimulation electrode from the ablation electrode.

Also provided in accordance with aspects of the present disclosure is an ablatio device including an elongated shaft extending distally from a handle, an electrode, and a retractable sleeve. The electrode is disposed on a distal portion of the elongated shaft and is configured to deliver electrosurgical energy to tissue. The retractable sleeve is disposed on the elongated shaft and is configured to be moved along the elongated shaft relative to the electrode to adjust a surface area of the electrode that is exposed during delivery of the electrosurgical energy to the tissue.

In aspects of the present disclosure, the electrode is a monopolar electrode.

### BRIEF DESCRIPTION OF THE DRAWINGS

Aspects of the disclosure are described herein with reference to the accompanying drawings, wherein:

FIG. 1 is a schematic illustration of an ablation system, in accordance with aspects of the present disclosure;

FIG. 2 is an enlarged view of the indicated area of detail shown in FIG. 1, in accordance with aspects of the present disclosure;

FIG. 3 is an enlarged view of the indicated area of detail shown in FIG. 1, in accordance with an alternative embodiment of the present disclosure;

FIG. 4 is an enlarged view of the indicated area of detail shown in FIG. 1, in accordance with yet another alternative embodiment of the present disclosure; and

FIG. 5 is a schematic illustration of an exemplary robotic surgical system configured for use with the ablation system of FIG. 1, in accordance with aspects of the present disclosure.

### DETAILED DESCRIPTION

Embodiments of the disclosure are now described in detail with reference to the drawings in which like reference numerals designate identical or corresponding elements in each of the drawings. Aspects of the various disclosed embodiments may be combined in any manner consistent with the functionality of the apparatus and/or method disclosed herein. As used herein, the term “clinician” refers to a doctor, a clinician, a nurse, or any other care provider and may include support personnel. Throughout this description, the term “proximal” will refer to the portion of the device or component thereof that is closer to the clinician and the term “distal” will refer to the portion of the device or component thereof that is farther from the clinician. As used herein, the term “exemplary” does not necessarily mean “preferred” and may simply refer to an example unless the context clearly indicates otherwise.

Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Additionally, in the drawings and in the description that follows, terms such as front, rear, upper, lower, top, bottom, and similar directional terms are used simply for convenience of description and are not intended to limit the disclosure. In the following description, well-known functions or constructions are not described in detail to avoid obscuring the disclosure in unnecessary detail.

In thyroid ablation procedures there is a risk of damaging nerves near the thyroid with heat from the ablating electrode during the ablation cycle. The present disclosure incorporates nerve stimulation into an ablation device (e.g., an ablation handpiece), which allows the surgeon to confirm sufficient distance of the ablating electrode from the nerves in the region of the nodule/tumor prior to initiating ablation. The ablation handpiece may generally include a handle and an elongated shaft extending from the handle. The stimulation signal is delivered at a controlled intensity (e.g., amplitude) to produce an expected dimensional spread of a signal in the tissue greater than the expected thermal spread from the ablation cycle. The stimulation signal is delivered to the tissue via a stimulation electrode disposed on the elongated shaft of the ablation device. If the stimulation signal is not picked up in the thyroid musculature where monitoring electrodes are located, then one would project that the ablating electrode is sufficiently distant from the nerve and that the ablation cycle could be completed with an acceptable level of risk.

One potential problem is that ablation electrodes are offered in a variety of lengths to accommodate surgeon preference and/or to address differing sizes of nodules/tumors. The reason this may be problematic is that the strength (or spread) of the stimulation signal delivered to the tissue is dependent on the surface area of the electrode delivering that energy. A longer electrode with more surface area will produce a more diffuse signal/energy level in the tissue. This is referred to as shunting. This shunting or changing of energy level in tissue impacts the predictability and consistency of energy spread in the tissue such that a stimulation signal of a given intensity from a 5 mm electrode, e.g., would be significantly different than a signal delivered by a 10 or 15 mm electrode (all common lengths of ablation electrodes). This results in a loss of predictability of the distance in which the stimulation signal spreads and thus, predictability of safe distance from the ablating electrode to the nerve.

A solution to the above-noted technical problem is to configure the ablation device such that the elongated shaft includes an ablation electrode and a defined, separate stimulation electrode that is consistent in surface area and in location on the elongated shaft of the ablation handpiece. The stimulation electrode may generally be located at the distal tip portion of the elongated shaft since that is the leading part of the elongated shaft, thereby allowing response from the nerve/muscle to be registered early on as the elongated shaft is being inserted into the tissue. The stimulating electrode may be separated longitudinally/axially from the ablating electrode by a dielectric ring of non-conductive material (e.g., ceramic, plastic, polyamide, nylon, a polymer, polyamide bioplastic, polyethylene terephthalate (PET), polyether ether ketone (PEEK), polytetrafluoroethylene (PTFE), or the like) to electrically isolate the ablation and stimulation electrodes from each other along the shaft. Internally within the elongated shaft, the stimulating electrode and the ablating electrode are electrically isolated from each other with wire coatings and suitable dielectric materials disposed on individual electrical leads that electrically couple the ablation electrode to an electrosurgical (e.g., RF) generator and the stimulation electrode to a stimulation signal generator. The electrosurgical generator and the stimulation signal generator may be disposed within a common enclosure (e.g., housing) or each may be disposed within its own individual enclosure and separate from each other. In the scenario where the electrosurgical generator and the stimulation signal generator are disposed within a common enclosure, the electrical leads may be electrically coupled to suitable input interfaces on the common enclosure to ensure electrical coupling of the stimulation electrode and the ablation electrode to the proper generator. The stimulation electrode may be located at the distal tip portion of the elongated shaft and may be consistent in size for all different lengths of the ablation electrode (e.g., 5 mm, 7 mm, 10 mm, 15 mm, etc.). This type of construction allows for any length of ablation electrode to be produced while not interfering with the stimulation signal delivery, thereby assuring a predictable spread of the signal given similar tissue properties. Moreover, the above-noted construction serves to avoid shunting of the stimulation signal through tissue, thereby preserving the effectiveness and predictability of the stimulation properties.

In aspects of the present disclosure, the dielectric ring may serve to isolate two or more poles in a bipolar configuration of the ablation device. For example, the stimulation electrode may be bipolar and the ablation electrode may be monopolar. In this scenario, the ablation electrode may be activated during ablation and the stimulation electrode may be deactivated during ablation. In other aspects of the present disclosure, both the stimulation electrode and the ablation electrode may be bipolar.

In some aspects of the present disclosure, the ablation device may include a single monopolar electrode and a sleeve that may be slid over an outer surface of the elongated shaft of the ablation device to selectively expose varying lengths of the monopolar electrode. In this scenario, the monopolar electrode may be used for stimulating and ablation. During stimulation, the sleeve serves to reduce an exposed surface area of the monopolar electrode such that only a distal tip portion of the monopolar electrode (at the distal tip portion of the elongated shaft) is exposed during stimulation. With the elongated shaft inserted into the target, the clinician may slide or retract the sleeve proximally along the elongated shaft to expose the monopolar electrode, thereby initiating ablation. In this scenario, the clinician may choose how much to retract the sleeve along the elongated shaft in accordance with what length of the monopolar electrode is to be exposed. In this way, electrode construction is simplified and the ablation device effectively includes different size ablation electrodes (e.g., 5 mm, 7 mm, 10 mm, 15 mm, etc.) consolidated in one device without the need to switch ablation devices to obtain the desired ablation electrode size.

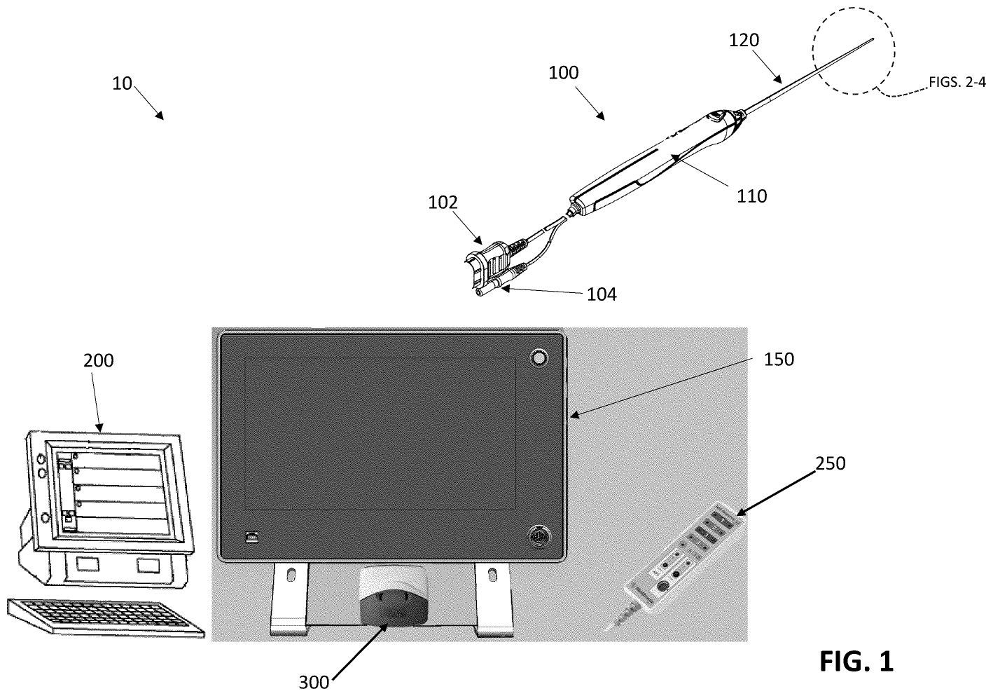

With reference to FIG. 1, an ablation system 10 according to aspects of the present disclosure is shown. The ablation system 10 generally includes an ablation device 100, an electrosurgical energy source, e.g., generator 150, a nerve monitoring unit 200, and an optional patient interface unit 250 that is connectable to the nerve monitoring unit 200 and the ablation device 100. Optionally, the patient interface unit 250 may be configured to be coupled to and to communicate with the generator 150. It should be understood that the generator 150 and the nerve monitoring unit 200 may be separate units, as shown in FIG. 1, or may be combined into a single unit (e.g., within a common enclosure) that includes the below-described functionality of both units. In this scenario, the need for the patient interface unit 250 may be obviated and the combined unit including the functionality of the generator 150 and the nerve monitoring unit 200 may include a user interface having a display screen that displays, for example, ablation status, stimulation status, ablation controls, and/or stimulation controls.

Optionally, the system 10 may include a fluid pump 300 configured to supply fluid (e.g., via inflow fluid tubing) to the ablation device 100 to cool the elongated shaft 120 during an ablation procedure. Likewise, the ablation device 100 may be configured to return fluid (e.g., via outflow fluid tubing) to the fluid pump 300 and/or a suitable container.

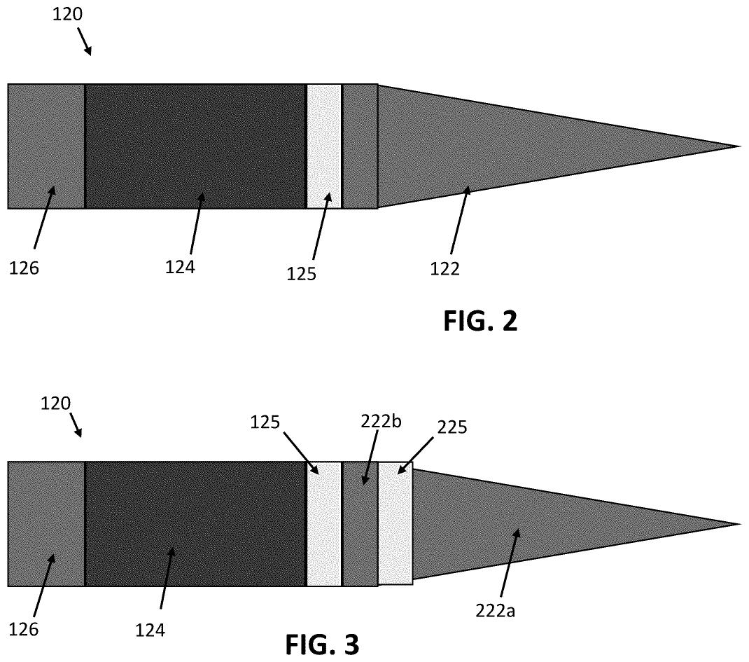

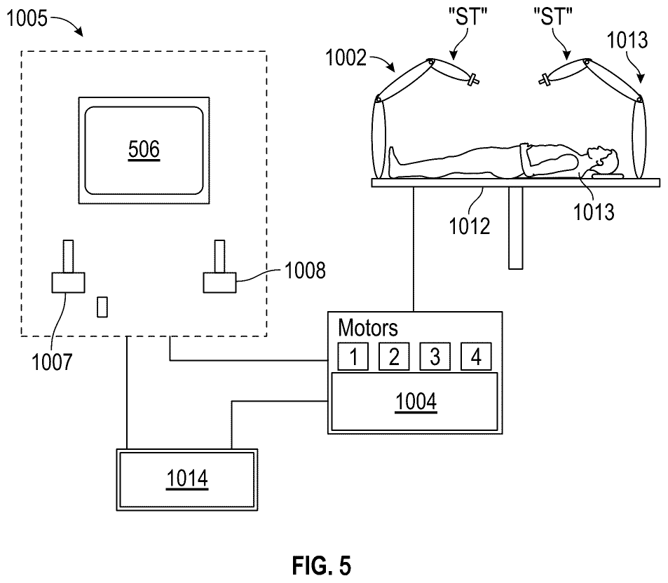

The ablation device 100 generally includes a handle 110 and an elongated shaft 120 extending distally from the handle 110 and having a tapered distal tip configured to pierce tissue. The handle 110 is configured to be gripped and manipulated by a clinician during an ablation procedure, although non-handle configurations are also contemplated, e.g., for mounting the ablation device 100 and/or attaching the ablation device 100 to a surgical robot arm (see FIG. 5). As detailed below with respect to FIG. 2, the elongated shaft 120 includes one or more electrodes configured to ablate tissue and one or more electrodes configured to stimulate nerves of the patient for the purpose of confirming sufficient distance of the one or more ablation electrodes from nerves in the region of the nodule/tumor prior to initiating ablation.

Referring to FIG. 2, an enlarged view of the indicated area of detail shown in FIG. 1 is shown and includes a distal portion of the elongated shaft 120 of the ablation device 100. The ablation device 100 includes a stimulation electrode 122 disposed at a distal tip portion of the elongated shaft 120 and an ablation electrode 124 disposed on the elongated shaft 120 proximal to the stimulation electrode 122. An electrically-insulative ring 125 of non-conductive material surrounds the elongated shaft 120 and is disposed between the stimulation electrode 122 and the ablation electrode 124 such that the stimulation electrode 122 and the ablation electrode 124 are separated axially along the elongated shaft 120 by the electrically-insulative ring 125 and electrically isolated from each other. A dielectric sleeve 126 is disposed on the elongated shaft 120 proximal to the ablation electrode 124 and serves to electrically insulate the elongated shaft 120 from the ablation electrode 124. In aspects of the present disclosure, stimulation electrode 122 may instead be a second ablation electrode in a bipolar configuration with the ablation electrode 124. In this scenario, the electrically-insulative ring 125 may instead be a stimulation electrode disposed between the two ablation electrodes 122, 124 and configured to deliver electrical stimulation to the nerves of the patient.

Referring back to FIG. 1, a generator connector 102 is coupled to a proximal end portion of the ablation device 100 via a suitable electrical cable and is configured to electrically couple the ablation electrode 124 of the ablation device 100 to the generator 150 (e.g., via insulated wiring extending through an internal lumen of the elongated shaft 120) to enable the generator 150 to deliver electrosurgical energy (e.g., RF energy) to the ablation electrode 124. A stimulation connector 104 is also coupled to a proximal end portion of the ablation device 100 via a suitable electrical cable and is configured to connect to an input of the patient interface unit 250 to electrically couple the stimulation electrode 122 of the ablation device 100 to the nerve monitoring unit 200 (e.g., via insulated wiring extending through an internal lumen of the elongated shaft 120) for delivering electrical stimulation to the nerves of the patient via the stimulation electrode 122. Alternatively, in scenarios where the patient interface unit 250 is not used, the stimulation electrode 122 may be directly electrically connected to the nerve monitoring unit 200 via the stimulation connector 104 and suitable insulated wiring extending through an internal lumen of the elongated shaft 120. Moreover, in scenarios where the generator 150 and the nerve monitoring unit 200 are combined into a single unit, as mentioned previously, the stimulation electrode 122 may be directly electrically connected to the single unit via the stimulation connector 104, thereby obviating the need for the patient interface unit 250. The handle 110 of the ablation device 100 may include suitable controls (e.g., buttons, switches, dials, or the like) to enable the clinician to control delivery of stimulation signals communicated from the nerve monitoring unit 200 to the stimulation electrode 122, thereby controlling the amount of electrical stimulation (e.g., voltage) being output by the stimulation electrode 122 during a procedure.

In aspects of the present disclosure, the ablation device 100 and the patient interface unit 250 are configured to be used in a sterile field in which the patient is positioned during an ablation procedure. The sterile field is located away from a non-sterile field in which the generator 150 and the nerve monitoring unit 200 are used. The patient interface unit 250 serves to couple to the ablation device 100 via the stimulation connector 104 within the sterile field for electrically coupling the stimulation electrode 122 to the nerve monitoring unit 200 in the non-sterile field.

The nerve monitoring unit 200 may include a display screen and one or more input devices (e.g., keyboard, mouse, touch screen, knob, button, audio input device, visual input device, tactile input device, etc.). The nerve monitoring unit 200 may further include a processor and a memory (not shown). It is understood that the processor may access the memory to execute instructions stored thereon or access other data on the memory. The memory may include a physical memory, such as a spinning hard disk drive, solid state memory, or other appropriate types of memory. Further, the memory may not be incorporated into the nerve monitoring unit 200, but may be accessed by the processor, such as via a communications network. The processor may be a general purpose processor that is operable to execute instructions for generating a selected output. The processor may further include onboard memory. Moreover, the processor may include a specific purpose processor such as an application specific integrated circuit (ASIC). Accordingly, the processor may execute instructions stored on memory, which may be a non-transitory memory, to provide an output for display on the display screen of the nerve monitoring unit 200.

The nerve monitoring unit 200 may further include a stimulation generator (not shown). The stimulation generator may be configured to generate a voltage based upon control by the processor. The processor may execute instructions of a program stored on the memory such that the nerve monitoring unit 200 may be operated to generate a stimulation at or with the stimulation electrode 122 of the ablation device 100 for monitoring nerves (e.g., the RLN and/or the vagus nerve) during various procedures such as, for example, a thyroidectomy or other thyroid surgery. Monitoring of the RLN may include placing one or more conductive electrodes (not shown) in contact with selected portions of a patient, e.g., via electrodes affixed to an exterior surface of a nerve monitoring endotracheal tube (not shown) electrically coupled to the nerve monitoring unit 200 and/or surface electrodes affixed to select portions of the patient's skin and used to monitor muscle response to a voltage provided to the patient's nerves via the stimulation electrode 122 to stimulate nerve activity. Monitoring of the RLN may alternatively or additionally include using needle or hook-wire electrodes (not shown) inserted into the muscles of the patient's neck. In some aspects of the present disclosure, surface electrodes, needle electrodes, and/or hook-wire electrodes may be used for awake patients and the esophageal tube may be used for patients under general anesthesia. In aspects of the present disclosure, the nerve monitoring unit 200 and the generator 150 may be combined into a single unit that includes the functionality of both units.

Various parameters for the electrical stimulation delivered by the nerve monitoring unit 200 and/or the patient interface unit 250, including current amplitude, pulse width, and repetition, are selectable and may be selected via the one or more input devices of the nerve monitoring unit 200. The patient interface unit 250 is capable of delivering electrical stimulation having a current amplitude in the range of about 0 to 30 mA to evoke electromyography (EMG) activity in the patient. The patient interface unit 250 includes a plurality of monitoring channels for the connection of monitoring or recording electrodes to be applied to the patient to detect EMG activity.

In aspects of the present disclosure, the generator 150 may include one or more processors and one or more processor-readable media (e.g., memory) storing instructions. The instructions may be executed by the processor, which may include one or more digital signal processors (DSPs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements.

In one or more examples, the described techniques may be implemented in hardware, software, firmware, or any combination thereof. If implemented in software, the functions may be stored as one or more instructions or code on a computer-readable medium and executed by a hardware-based processing unit. Processor-readable media may include non-transitory processor-readable media, which corresponds to a tangible medium such as data storage media (e.g., RAM, ROM, EEPROM, flash memory, or any other medium that can be used to store desired program code in the form of instructions or data structures and that can be accessed by a processor).

Commercially available ablation systems for use with the present disclosure include, for example, the Cool-tip™ RF Ablation System and the Accurian™ RF Ablation Platform both available from Medtronic plc of Dublin, Ireland. Although aspects of the present disclosure may be described in terms of using RF energy to ablate tissue, such description should not be considered limiting. It is contemplated that the ablation device 100 and the generator 150 of the present disclosure may be configured for use with other suitable forms of ablation energy such as, for example, microwave, laser, ultrasonic, and/or cryogenics. In aspects of the present disclosure, the ablation device 100 and the generator 150 may be configured to use pulsed field ablation (PFA), which is a non-thermal method of ablating tissue using pulsed electric fields. The generator 150 may be configured to operate in one or both of a monopolar configuration or a bipolar configuration and includes suitable outputs for delivering electrosurgical energy to the ablation device 100 and/or suitable inputs for returning electrosurgical energy to the generator 150.

In aspects of the present disclosure, the ablation electrode 124 may be configured as a monopolar electrode that contacts and ablates tissue. In this scenario, the ablation system 10 may include a remote return electrode or return pad (not shown) coupled to the generator 150 and configured to be adhered to a patient. In other aspects of the present disclosure, the ablation electrode 124 may be in a bipolar configuration utilizing two electrodes disposed on the elongated shaft 120 of the ablation device 100 to deliver energy to tissue disposed between the two electrodes. In this scenario, the two electrodes may be axially spaced (e.g., 5-10 mm) from one another along the elongated shaft 120 and may be electrically isolated from one another by an insulative material (e.g., an electrically-insulative ring similar to insulative ring 125) disposed on the elongated shaft 120 between the two electrodes.

In aspects of the present disclosure, the stimulation electrode 122 may be configured as a monopolar electrode. In other aspects of the present disclosure, the stimulation electrode 122 may be in a bipolar configuration utilizing two electrodes disposed on the elongated shaft 120 of the ablation device 100. In this scenario, the two electrodes may be axially spaced (e.g., between about 5 mm and about 10 mm or between about 1 mm and about 5 mm) from one another along the elongated shaft 120 and may be electrically isolated from one another by an insulative material (e.g., an insulative ring similar to insulative ring 125) disposed on the elongated shaft 120 between the two electrodes. For example, FIG. 3 shows a bipolar stimulation electrode configuration having a pair of stimulation electrodes 222a, 222b disposed on the distal tip portion of the elongated shaft 120 and axially spaced from each other with an insulative ring 225 disposed between the stimulation electrodes 222a, 222b to electrically isolate the stimulation electrodes 222a, 222b from one another.

In aspects of the present disclosure, the bipolar configuration of the stimulation electrode 124 includes a single anodal electrode and a single cathodal electrode. Electrical current flowing through the two electrodes may be directly or indirectly applied to a nerve to stimulate the nerve. A negative electrical current may be applied to the nerve via the cathodal electrode (referred to as a cathode or negative electrode). The nerve resists excitation at the anodal electrode (referred to as an anode or positive electrode). This is a result of negative current from the cathode reducing voltage outside a neuronal cell membrane of the nerve, causing depolarization and an action potential. The anode injects positive current outside the neuronal cell membrane, which leads to hyperpolarization. Preferential cathodal stimulation refers to a reduced amount of current (one third to one quarter) needed to elicit a motor response of a muscle when the cathode is used as the stimulating electrode. The amount of current applied when the cathode is used is less than an amount of current needed to elicit a motor response of a muscle when the anode is used as the stimulating electrode. In order to stimulate a nerve using the cathode: the cathode may be attached to a stimulating needle or catheter; and the anode may be used as a current returning electrode and be attached to or in contact with the skin of the patient via a return wire.

In aspects of the present disclosure, the ablation electrode 124 may be monopolar and the stimulation electrode 122 may be bipolar (e.g., including two electrodes separated from each other) with both electrodes activated during ablation or, alternatively, with the ablation electrode 124 activated and the stimulation electrode 122 deactivated during ablation. It should be understood, however, that any combination of monopolar and bipolar configurations of the ablation electrode 124 and the stimulation electrode 122 is possible and contemplated as part of the present disclosure. In operation, the stimulation electrode 122 may remain activated during insertion of the elongated shaft 120 into tissue until the ablation electrode 124 is activated by the clinician (e.g., via suitable controls on the handle 110 and/or via a footswitch coupled to the generator 150), which serves to automatically deactivate the stimulation electrode 122 by terminating delivery of stimulation signals from the nerve monitoring unit 200 and/or the patient interface unit 250 to the stimulation electrode 122. Deactivation of the ablation electrode 124 may serve to automatically re-activate the stimulation electrode 122.

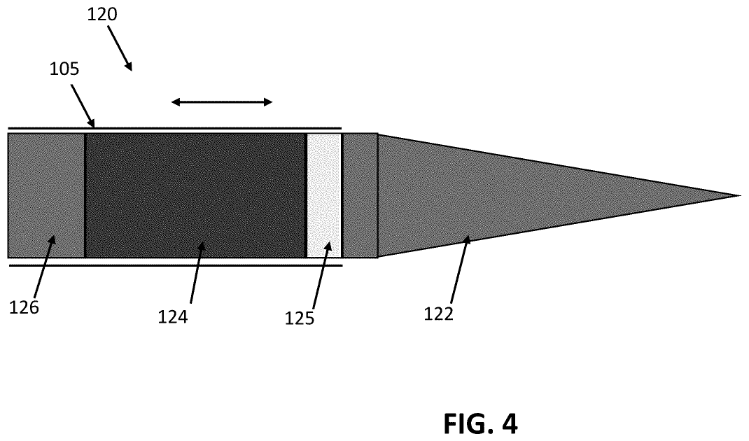

With reference to FIG. 4, the ablation device 100, in accordance with aspects of the present disclosure, may include a sleeve 105 disposed on an outer surface of the elongated shaft 120. The sleeve 105 may be suitably lubricious to enable sliding of the sleeve 105 relative to the elongated shaft 120 proximally and distally along a longitudinal axis of the elongated shaft 120 to selectively expose and cover the stimulation electrode 122 and the ablation electrode 124. By way of example, the sleeve 105 may be manipulated such that only the stimulation electrode 122 is exposed during stimulation. With the elongated shaft 120 inserted into the target, the clinician may then choose to slide or retract the sleeve 105 proximally along the elongated shaft 120 to expose the ablation electrode 124 to initiate ablation. In this scenario, the ablation electrode 124 may be activated during stimulation but prevented from ablating tissue, due to being covered by the sleeve 105, until the sleeve 105 is retracted proximally to expose the ablation electrode 124 or to expose at least a portion of the ablation electrode 124. In operation, the clinician may choose how much of the ablation electrode 124 is to be exposed by the sleeve 105 in accordance with what length of ablation electrode is suitable for a given procedure. Thus, use of the sleeve 105 in the above-noted configuration allows the ablation device 100 to effectively include different size ablation electrodes (e.g., 5 mm, 7 mm, 10 mm, 15 mm, etc.) consolidated in one device.

Turning now to FIG. 5, a robotic surgical system 1000 configured for use in accordance with the present disclosure is shown. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a clinician, e.g., a clinician, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and a mounted device which may be, for example, a surgical tool “ST.” The surgical tools “ST” may include, for example, the ablation device 100 of the present disclosure, thus providing any of the above-detailed functionality on a robotic surgical system 1000.

Robot arms 1002, 1003 may be driven by electric drives, e.g., motors, connected to control device 1004. The motors, for example, may be rotational drive motors configured to provide rotational inputs to accomplish a desired task or tasks. Control device 1004, e.g., a computer, may be configured to activate the motors, in particular by means of a computer program, in such a way that robot arms 1002, 1003, and, thus, their mounted surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Control device 1004, more specifically, may control one or more of the motors based on rotation, e.g., controlling to rotational position using a rotational position encoder (or Hall effect sensors or other suitable rotational position detectors) associated with the motor to determine a degree of rotation output from the motor and, thus, the degree of rotational input provided. Alternatively or additionally, control device 1004 may control one or more of the motors based on torque, current, or in any other suitable manner.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. An ablation system, comprising:
an ablation device configured to be inserted into an ablation target, the ablation device including:
an elongated shaft extending distally from a handle;
a stimulation electrode disposed on a distal tip portion of the elongated shaft, the stimulation electrode configured to electrically stimulate nerves;
an ablation electrode disposed on the elongated shaft and axially spaced proximally from the stimulation electrode, the ablation electrode configured to deliver electrosurgical energy to tissue; and
an insulative ring surrounding the elongated shaft and disposed between the stimulation electrode and the ablation electrode;
an electrosurgical generator configured to deliver electrosurgical energy to the ablation electrode of the ablation device; and
a nerve monitoring unit configured to deliver electrical stimulation signals to the stimulation electrode of the ablation device.

2. The ablation system according to claim 1, further comprising a patient interface unit electrically coupleable to the nerve monitoring unit and configured to electrically couple the stimulation electrode to the nerve monitoring unit.

3. The ablation system according to claim 1, wherein the electrosurgical generator and the nerve monitoring unit are disposed within a common enclosure.

4. An ablation device, comprising:
an elongated shaft extending distally from a handle;
a stimulation electrode disposed on a distal tip portion of the elongated shaft, the stimulation electrode configured to electrically stimulate nerves;
an ablation electrode disposed on the elongated shaft and axially spaced proximally from the stimulation electrode, the ablation electrode configured to deliver electrosurgical energy to tissue; and
an insulative ring surrounding the elongated shaft and disposed between the stimulation electrode and the ablation electrode to electrically isolate the stimulation electrode from the ablation electrode.

5. An ablation device, comprising:
an elongated shaft extending distally from a handle;
an electrode disposed on a distal portion of the elongated shaft and configured to deliver electrosurgical energy to tissue; and
a retractable sleeve disposed on the elongated shaft and configured to be moved along the elongated shaft relative to the electrode to adjust a surface area of the electrode that is exposed during delivery of the electrosurgical energy to the tissue.

6. The ablation device according to claim 5, wherein the electrode is monopolar.
